(19) United States
(12) Patent Application Publication
(10) Pub. No.: US 2013/0017214 A1
(43) Pub. Date: Jan. 17, 2013

(54) BACTEREMIA-ASSOCIATED ANTIGEN FROM STAPHYLOCOCCUS AUREUS

(71) Applicant: Jonathan Edgeworth, London (GB)

(72) Inventor: Jonathan Edgeworth, London (GB)

(73) Assignee: GUY'S AND ST THOMAS'S NHS FOUNDATION TRUST, London (GB)

(21) Appl. No.: 13/509,272

(22) PCT Filed: Nov. 9, 2010
(86) PCT No.: PCT/GB2010/002056

§ 371 Date: Oct. 1, 2012

(30) Foreign Application Priority Data
Nov. 10, 2009 (GB) 0919690.8

(51) Int. Cl. C07K 14/31; C40B 30/04; G01N 33/569; A61P 31/04; A61K 39/085; C07K 16/12; A61P 37/04; C12Q 1/68; C07K 19/00

(52) U.S. Cl. 424/190.1; 435/6.15; 435/6.11; 435/6.12; 506/9; 435/7.1; 530/350; 530/387.9

(57) ABSTRACT

The BAA antigen of Staphylococcus aureus was identified in a highly bacteremic MRSA strain that has enhanced in vitro binding to fibronectin and other extracellular matrix proteins compared with other endemic and related strains. BAA is a phage-encoded surface-expressed adhesin which binds fibronectin in vitro. It may be responsible for the enhanced catheter-related bacteremic phenotype of MRSA strains and is thus useful as a vaccine target to prevent MRSA bacteremia.

This application claims the benefit of United Kingdom patent application 0919690.8, filed Nov. 10, 2009, the complete contents of which are incorporated herein by reference for all purposes.

### TECHNICAL FIELD

This invention relates to compositions for immunising against Staphylococcus aureus.

### BACKGROUND ART

S. aureus is a Gram-positive spherical bacterium. Annual US mortality exceeds that of any other infectious disease, including HIV/AIDS, and S. aureus is the leading cause of bloodstream, lower respiratory tract, skin & soft tissue infections. A particular cause of concern is MRSA (methicillin-resistant S. aureus) which is resistant to most antibiotics.

There is currently no authorised S. aureus vaccine. Reference 1 reports that the “V710” vaccine from Merck and Intercell is undergoing a phase 2/3 trial on patients undergoing cardiothoracic surgery. The V710 vaccine is based on a single antigen, IsdB [2], an iron-sequestering cell-surface protein.

There remains a need to identify further and improved antigens for use in S. aureus vaccines, and in particular for vaccines which are useful against MRSA strains.

### DISCLOSURE OF THE INVENTION

The “TW” strain of MRSA is in ST-239 and has an enhanced ability to adhere to vascular catheters and cause bacteremia in humans [3]. It also has enhanced in vitro binding to a range of extracellular matrix proteins including fibronectin, compared with other endemic and related strains. It also invades endothelial cells better than endemic or related strains. The inventors have identified a bacteremia-associated antigen “BAA” in TW. BAA is a phage-encoded surfaced-expressed adhesin which demonstrates in vitro binding to fibronectin and so may be responsible for the enhanced catheter-related bacteremic phenotype of MRSA strains. It may thus be useful as a vaccine target e.g. to prevent MRSA-caused bacteremia, including against type ST239 strains.

The invention provides a BAA antigen for use in immunising against S. aureus disease and/or infection.

The invention also provides a fusion protein comprising a BAA antigen polypeptide sequence and at least one further S. aureus antigen polypeptide sequence. The fusion protein is useful as an active ingredient in an immunogenic composition for immunising against S. aureus disease.

The invention also provides an immunogenic composition comprising a BAA antigen and at least one further S. aureus antigen. The composition is useful for immunising against S. aureus disease.

The invention also provides an immunogenic composition comprising a BAA antigen and at least one non-S. aureus antigen. The composition is useful for immunising against a range of diseases.

The invention also provides an immunogenic composition comprising a combination of: (1) a BAA antigen; and (2) an adjuvant.

The invention also provides an anti-BAA antibody. The antibody is useful for treating and/or preventing S. aureus disease e.g. alone or in combination with another therapy, such as an antibiotic.

The invention provides a method for detecting the presence or absence of a S. aureus bacterium in a sample, comprising detecting a BAA antigen, or nucleic acid encoding a BAA antigen, in the sample.

BAA Antigen

The BAA antigen has previously been seen in S. epidermidis and has been called SesI [4,5], SE1654 [6] or SesD [7]. In S. aureus strains, however, the BAA antigen has previously been reported as absent [6]. Work on the S. epidermidis antigen has confirmed that the antigen is a marker of invasive capacity (and likely a virulence factor), is immunogenic and can elicit opsonophagocytic activity against the bacterium. The same properties can be expected for the same antigen in S. aureus. Moreover, the BAA antigen in S. aureus has been confirmed to have fibronectin-binding activity, confirming its surface exposure and functional expression, thus again supporting the view that the S. aureus protein should share the properties already seen for the S. epidermidis protein.

A BAA antigen of the invention may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1; and/or (b) comprising a fragment of at least ‘n’ consecutive amino acids of SEQ ID NO: 1, wherein ‘n’ is 7 or more (e.g. 8, 10, 12, 14, 16, 18, 20, 25, 30, 35, 40, 50, 60, 70, 80, 90, 100, 150, 200, 250 or more). These BAA sequences include variants of SEQ ID NO: 1. Preferred fragments of (b) comprise an epitope from SEQ ID NO: 1. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 1 while retaining at least one epitope of SEQ ID NO: 1.

Thus, for instance, polypeptides used with the invention may, compared to SEQ ID NO: 1, include one or more (e.g. 1, 2, 3, 4, 5, 6, 7, 8, 9, etc.) amino acid substitutions, such as conservative substitutions (i.e. substitutions of one amino acid with another which has a related side chain). Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, etc.) single amino acid deletions relative to SEQ ID NO: 1. The polypeptides may also include one or more (e.g. 1, 2, 3, 4, 5, 6, 7, 8, 9, etc.) insertions (e.g. each of 1, 2, 3, 4 or 5 amino acids) relative to SEQ ID NO: 1.

Similarly, a BAA antigen may comprise an amino acid sequence that:
(a) is identical (i.e. 100% identical) to SEQ ID NO: 1;
(b) shares sequence identity (e.g. 80%, 85%, 90%, 91%, 92%, 93%, 94%, 95%, 96%, 97%, 98%, 99%, 99.5% or more) with SEQ ID NO: 1;
(c) has 1, 2, 3, 4, 5, 6, 7, 8, 9 or 10 (or more) single amino acid alterations (deletions, insertions, substitutions), which may be at separate locations or may be contiguous, as compared to SEQ ID NO: 1; and/or
(d) when aligned with SEQ ID NO: 1 using a pairwise alignment algorithm, each moving window of x amino acids from N-terminus to C-terminus (such that for an alignment that extends to p amino acids, where p>x, there are p-x+1 such windows) has at least x·y identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x·y is not an integer then it is rounded up to the nearest integer. The preferred pairwise alignment algorithm is the Needleman-Wunsch global alignment algorithm [8], using default parameters (e.g. with Gap opening penalty=10.0, and with Gap extension penalty=0.5, using the EBLOSUM62 scoring matrix). This algorithm is conveniently implemented in the needle tool in the EMBOSS package [9].

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus. N-terminus truncation can remove leader peptides e.g. to facilitate recombinant expression in a heterologous host. C-terminus truncation can remove anchor sequences e.g. to facilitate recombinant expression in a heterologous host.

In some embodiments, the invention provides a fusion protein comprising a BAA antigen polypeptide sequence and at least one further antigen (preferably a S. aureus antigen) polypeptide sequence. Thus a single polypeptide chain can provide two distinct antigenic functions. Fusion proteins consisting of BAA and amino acid sequences from two, three, four, five, six, seven, eight, nine, or ten further antigens are useful.

The fusion protein can be combined with conjugates or non-S. aureus antigens as described below.

Fusion proteins can be represented by the formula NH₂-A-{—X-L-}ₙ-B—COOH, wherein: X is an amino acid sequence of an antigen, as described above; L is an optional linker amino acid sequence; A is an optional N-terminal amino acid sequence; B is an optional C-terminal amino acid sequence; n is an integer of 2 or more (e.g. 2, 3, 4, 5, 6, etc.). At least one —X— moiety is a BAA antigen. Usually n is 2 or 3. When n is 2, the BAA sequence may be X₁ or X₂ i.e. the N-terminus or C-terminus antigen.

If a —X— moiety has a leader peptide sequence in its wild-type form, this may be included or omitted in the fusion protein. In some embodiments, the leader peptides will be deleted except for that of the —X— moiety located at the N-terminus of the fusion protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {—X-L-}, linker amino acid sequence -L- may be present or absent. For instance, when n=2 the fusion protein may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. His where n=3, 4, 5, 6, 7, 8, 9, 10 or more). Other suitable linker amino acid sequences will be apparent to those skilled in the art. A useful linker is GSGGGG (SEQ ID NO: 2) or GSGSGGGG (SEQ ID NO: 3), with the Gly-Ser dipeptide being formed from a BamHI restriction site, thus aiding cloning and manipulation, and the (Gly)₄ tetrapeptide being a typical poly-glycine linker. Other suitable linkers, particularly for use as the final Lₙ are ASGGGS (SEQ ID NO: 4) or a Leu-Glu dipeptide.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. His where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine e.g. Met-Ala-Ser, or a single Met residue.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more, such as SEQ ID NO: 5), or sequences which enhance protein stability. Other suitable C-terminal amino acid sequences will be apparent to those skilled in the art.

In general, when a polypeptide comprises a sequence that is not identical to SEQ ID NO: 1 (e.g. when it comprises a sequence with <100% sequence identity thereto, or when it comprises a fragment thereof), or when a polypeptide comprises antigen(s) in addition to the BAA sequence, it is preferred that the polypeptide can elicit an antibody which recognises the S. aureus protein having amino acid sequence SEQ ID NO: 1. Thus a BAA antigen or a fusion protein of the invention may (e.g. when administered to a human) elicit an antibody that recognises the wild-type protein encoded in the S. aureus genome as SEQ ID NO: 1.

Further S. aureus Antigens

In some embodiments an immunogenic composition comprises a BAA antigen in combination with at least one further S. aureus antigen. The further antigen(s) can be polypeptide and/or saccharide antigens.

Suitable polypeptide antigen(s) may be selected from the following (as disclosed and defined in references 10-17): immunodominant ABC transporter, laminin receptor, SsaA, SitC (also known as MntC), IsaA (also known as PisA or IssA), EbhA, EbhB, Aap, RAP (RNA III activating protein), FIG, EbpS, EFB, alpha toxin (hemolysin), SBI, IsdA, IsdB, SdrC, C1fA, FnbA, C1fB, coagulase, FnbB, MAP, HarA, autolysin glucosaminidase, autolysin amidase, Ebh, autolysin Ant, MRPII, SdrG, SdrE, SdrD, SasF, AhpC, AhpF, Collagen binding protein CAN, GehD lipase, Heparin binding protein HBP (17 kDa), Npase, ORF0594, ORF0657n, ORF0826, PBP4, Sai-1, SasK, SBI, SdrH, SSP-1, SSP-2, Vitronectin-binding protein.

Suitable saccharide antigen(s) include S. aureus capsular saccharide(s) and/or the S. aureus exopolysaccharide. A saccharide antigen may be conjugated to a carrier protein.

The exopolysaccharide of S. aureus is a poly-N-acetylglucosamine (PNAG). The saccharide may be a polysaccharide having the size that arises during purification of the exopolysaccharide from bacteria, or it may be an oligosaccharide achieved by fragmentation of such a polysaccharide e.g. size can vary from over 400 kDa to between 75 and 400 kDa, or between 10 and 75 kDa, or up to 30 repeat units. The saccharide moiety can have various degrees of N-acetylation and, as described in reference 18, the PNAG may be less than 40% N-acetylated (e.g. less than 35, 30, 20, 15, 10 or 5% N-acetylated; deacetylated PNAG is also known as dPNAG). Deacetylated epitopes of PNAG can elicit antibodies that are capable of mediating opsonic killing. The PNAG may or may not be O-succinylated e.g. on fewer than 25, 20, 15, 10, 5, 2, 1 or 0.1% of residues.

The capsular saccharide of a S. aureus may be a polysaccharide having the size that arises during purification of capsular polysaccharide from bacteria, or it may be an oligosaccharide achieved by fragmentation of such a polysaccharide. Capsular saccharides may be obtained from any suitable strain of S. aureus (or any bacterium having a similar or identical saccharide), such as from a type 5 and/or a type 8 S. aureus strain and/or a type 336 S. aureus strain. Most strains of infectious S. aureus contain either Type 5 or Type 8 capsular saccharides. Both have FucNAcp in their repeat unit as well as ManNAcA which can be used to introduce a sulfhydryl group for linkage. The repeating unit of the Type 5 saccharide is →4)-β-D-Man NAcA-(1→4)-α-L-FucNAc(3OAc)-(1→3)-β-D-FucNAc-(1→, whereas the repeating unit of the Type 8 saccharide is →3)-β-D-ManNAcA(4OAc)-(1→3)-α-L-FucNAc(1→3)-α-D-FucNAc(1→. The type 336 saccharide is a β-linked hexosamine with no O-acetylation [19,20] and is cross-reactive with antibodies raised against the 336 strain (ATCC 55804). A combination of a type 5 and a type 8 saccharide is typical, and a type 336 saccharide may be added to this pairing [21].

Thus, for example, a composition may include a BAA antigen and (a) a conjugate of a type 5 capsular saccharide and/or (b) a conjugate of a type 8 capsular saccharide.

The carrier moiety in conjugates will usually be a protein. Typical carrier proteins are bacterial toxins, such as diphtheria or tetanus toxins, or toxoids or mutants or fragments thereof. The CRM197 diphtheria toxin mutant [22] is useful. Other suitable carrier proteins include the N. meningitidis outer membrane protein complex [23], synthetic peptides [24,25], heat shock proteins [26,27], pertussis proteins [28,29], cytokines [30], lymphokines [30], hormones [30], growth factors [30], artificial proteins comprising multiple human CD4⁺ T cell epitopes from various pathogen-derived antigens [31] such as N19 [32], protein D from H. influenzae [33-35], pneumolysin [36] or its non-toxic derivatives [37], pneumococcal surface protein PspA [38], iron-uptake proteins [39], toxin A or B from C. difficile [40], recombinant P. aeruginosa exoprotein A (rEPA) [41], etc. In some embodiments the carrier protein is a S. aureus protein, such as BAA.

Where a composition includes more than one conjugate, each conjugate may use the same carrier protein or a different carrier protein.

Conjugates may have excess carrier (w/w) or excess saccharide (w/w). In some embodiments, a conjugate may include substantially equal weights of each.

The carrier molecule may be covalently conjugated to the carrier directly or via a linker. Direct linkages to the protein may be achieved by, for instance, reductive amination between the saccharide and the carrier, as described in, for example, references 42 and 43. The saccharide may first need to be activated e.g. by oxidation. Linkages via a linker group may be made using any known procedure, for example, the procedures described in references 44 and 45. A preferred type of linkage is an adipic acid linker, which may be formed by coupling a free —NH₂ group (e.g. introduced to a glucan by amination) with adipic acid (using, for example, diimide activation), and then coupling a protein to the resulting saccharide-adipic acid intermediate [46,47]. Another preferred type of linkage is a carbonyl linker, which may be formed by reaction of a free hydroxyl group of a saccharide CDI [48, 49] followed by reaction with a protein to form a carbamate linkage. Other linkers include β-propionamido [50], nitrophenyl-ethylamine [51], haloacyl halides [52], glycosidic linkages [53], 6-aminocaproic acid [54], ADH [55], C₄ to C₁₂ moieties [56], etc. Carbodiimide condensation can also be used [57].

PNAG conjugates may be prepared in various ways e.g. by a process comprising: a) activating the PNAG by adding a linker comprising a maleimide group to form an activated PNAG; b) activating the carrier protein by adding a linker comprising a sulfydryl group to form an activated carrier protein; and c) reacting the activated PNAG and the activated carrier protein to form a PNAG-carrier protein conjugate; or by a process comprising a) activating the PNAG by adding a linker comprising a sulfydryl group to form an activated PNAG; b) activating the carrier protein by adding a linker comprising a maleimide group to form an activated carrier protein; and c) reacting the activated PNAG and the activated carrier protein to form a PNAG-carrier protein conjugate; or by a process comprising a) activating the PNAG by adding a linker comprising a sulfydryl group to form an activated PNAG; b) activating the carrier protein by adding a linker comprising a sulfydryl group to form an activated carrier protein; and c) reacting the activated PNAG and the activated carrier protein to form a PNAG-carrier protein conjugate.

Non-S. aureus Antigens

In some embodiments an immunogenic composition comprises a BAA antigen in combination with at least one non-S. aureus antigen. Suitable non-staphylococcal antigens include, but are not limited to, antigens from bacteria associated with nosocomial infections. For example, the antigen may be from one of the following pathogens: S. epidermidis; Clostridium difficile; Pseudomonas aeruginosa; Candida albicans; and/or extraintestinal pathogenic Escherichia coli. Further suitable antigens for use in combination with a BAA antigen are listed on pages 33-46 of reference 58.

Polypeptides Used with the Invention

Polypeptides used with the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides used with the invention can be prepared by various means (e.g. recombinant expression, purification from cell culture, chemical synthesis, etc.). Recombinantly-expressed proteins are preferred, particularly for fusion proteins.

Polypeptides used with the invention are preferably provided in purified or substantially purified form i.e. substantially free from other polypeptides (e.g. free from naturally-occurring polypeptides), particularly from other staphylococcal or host cell polypeptides, and are generally at least about 50% pure (by weight), and usually at least about 90% pure i.e. less than about 50%, and more preferably less than about 10% (e.g. 5%) of a composition is made up of other expressed polypeptides. Thus the antigens in the compositions are separated from the whole organism with which the molecule is expressed.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included are, for example, polypeptides containing one or more analogs of an amino acid (including, for example, unnatural amino acids, etc.), as well as other modifications known in the art. Polypeptides can occur as single chains or associated chains.

The invention provides polypeptides comprising a sequence —P-Q- or -Q-P—, wherein: —P— is an amino acid sequence as defined above and -Q- is not a sequence as defined above i.e. the invention provides fusion proteins. Where the N-terminus codon of —P— is not ATG, but this codon is not present at the N-terminus of a polypeptide, it will be translated as the standard amino acid for that codon rather than as a Met. Where this codon is at the N-terminus of a polypeptide, however, it will be translated as Met. Examples of -Q- moieties include, but are not limited to, histidine tags (i.e. His, where n=3, 4, 5, 6, 7, 8, 9, 10 or more), maltose-binding protein, or glutathione-S-transferase (GST).

The invention also provides a process for producing a polypeptide of the invention, comprising the step of culturing a host cell transformed with nucleic acid of the invention under conditions which induce polypeptide expression.

Although expression of the polypeptides of the invention may take place in a Staphylococcus, the invention will usually use a heterologous host for expression (recombinant expression). The heterologous host may be prokaryotic (e.g. a bacterium) or eukaryotic. It may be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc. Compared to the wild-type S. aureus genes encoding polypeptides of the invention, it is helpful to change codons to optimise expression efficiency in such hosts without affecting the encoded amino acids.

The invention provides a process for producing a polypeptide of the invention, comprising the step of synthesising at least part of the polypeptide by chemical means.

Antibodies

Anti-BAA antibodies can be used for passive immunisation or for immunotherapy. Thus the invention provides an anti-BAA antibody for use in therapy. The invention also provides the use of such antibodies in the manufacture of a medicament. The invention also provides a method for treating a mammal comprising the step of administering an effective amount of an anti-BAA antibody of the invention. As described above for immunogenic compositions, these methods and uses allow a mammal to be protected against S. aureus infection and/or disease.

The term “antibody” includes intact immunoglobulin molecules, as well as fragments thereof which are capable of binding an antigen. These include hybrid (chimeric) antibody molecules [59, 60]; F(ab′)₂ and F(ab) fragments and Fv molecules; non-covalent heterodimers [61, 62]; single-chain Fv molecules (sFv) [63]; dimeric and trimeric antibody fragment constructs; minibodies [64, 65]; humanized antibody molecules [66-68]; and any functional fragments obtained from such molecules, as well as antibodies obtained through non-conventional processes such as phage display. Preferably, the antibodies are monoclonal antibodies. Methods of obtaining monoclonal antibodies are well known in the art. Humanised or fully-human antibodies are preferred.

Monoclonal antibodies are particularly useful in identification and purification of the individual polypeptides against which they are directed. Monoclonal antibodies of the invention may also be employed as reagents in immunoassays, radioimmunoassays (RIA) or enzyme-linked immunosorbent assays (ELISA), etc. In these applications, the antibodies can be labelled with an analytically-detectable reagent such as a radioisotope, a fluorescent molecule or an enzyme. The monoclonal antibodies produced by the above method may also be used for the molecular identification and characterization (epitope mapping) of polypeptides of the invention.

Antibodies of the invention are preferably provided in purified or substantially purified form. Typically, the antibody will be present in a composition that is substantially free of other polypeptides e.g. where less than 90% (by weight), usually less than 60% and more usually less than 50% of the composition is made up of other polypeptides.

Antibodies of the invention can be of any isotype (e.g. IgA, IgG, IgM i.e. an α, γor μ heavy chain), but will generally be IgG. Within the IgG isotype, antibodies may be IgG1, IgG2, IgG3 or IgG4 subclass. Antibodies of the invention may have a κ or a λ light chain.

An anti-BAA antibody can be administered to a patient in conjunction with an antibiotic. The antibiotic can be administered in admixture with the antibody (and thus a composition of the invention can include an antibiotic which is active against a staphylococcus) or separately.

Anti-BAA antibodies are particularly suitable for infusion to adults or neonates at short term risk of bacteremia due to strains of S. aureus (or S. epidermidis) carrying this or potentially structurally related adhesins e.g. for unexpected or unpredictable admissions in emergency situations, such as intensive care units during an outbreak or where such strains are known to be endemic. The antibodies can also be used as adjunctive therapy with antibiotics for treatment of S. aureus bacteremia involving foreign body or deep-seated infections.

Nucleic Acids

The invention also provides nucleic acid encoding polypeptides and fusion polypeptides of the invention. It also provides nucleic acid comprising a nucleotide sequence (e.g. SEQ ID NO: 8) that encodes one or more polypeptides or fusion polypeptides of the invention.

The invention also provides nucleic acid comprising nucleotide sequences having sequence identity to such nucleotide sequences. Identity between sequences is preferably determined by the Smith-Waterman homology search algorithm as described above. Such nucleic acids include those using alternative codons to encode the same amino acid.

The invention also provides nucleic acid which can hybridize to these nucleic acids. Hybridization reactions can be performed under conditions of different “stringency”. Conditions that increase stringency of a hybridization reaction of widely known and published in the art (e.g. page 7.52 of reference 255). Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art (e.g. see refs 69, 255, 257, etc.].

In some embodiments, nucleic acid of the invention hybridizes to a target under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

The invention includes nucleic acid comprising sequences complementary to these sequences (e.g. for antisense or probing, or for use as primers).

Nucleic acids of the invention can be used in hybridisation reactions (e.g. Northern or Southern blots, or in nucleic acid microarrays or ‘gene chips’) and amplification reactions (e.g. PCR, SDA, SSSR, LCR, TMA, NASBA, etc.) and other nucleic acid techniques.

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other staphylococcal or host cell nucleic acids, generally being at least about 50% pure (by weight), and usually at least about 90% pure. Nucleic acids of the invention are preferably staphylococcal nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

Nucleic acid amplification according to the invention may be quantitative and/or real-time.

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

Mutant Bacteria

The invention also provides a S. aureus bacterium in which a BAA antigen has been knocked out. Techniques for producing knockout bacteria are well known, and knockout S. aureus strains have been reported. A knockout mutation may be situated in the coding region of the gene or may lie within its transcriptional control regions (e.g. within its promoter). A knockout mutation will reduce the level of mRNA encoding the antigen to <1% of that produced by the wild-type bacterium, preferably <0.5%, more preferably <0.1%, and most preferably to 0%.

The invention also provides a S. aureus in which BAA antigen has a mutation which inhibits its activity. The gene encoding the antigen will have a mutation that changes the encoded amino acid sequence. Mutation may involve deletion, substitution, and/or insertion, any of which may be involve one or more amino acids.

The invention also provides a bacterium, such as a S. aureus bacterium, which hyper-expresses a BAA antigen.

The invention also provides a bacterium, such as a S. aureus bacterium, that constitutively expresses a BAA antigen. The invention also provides a staphylococcus comprising a gene encoding a BAA antigen, wherein the gene is under the control of an inducible promoter.

Immunogenic Compositions and Medicaments

Immunogenic compositions of the invention may be useful as vaccines. Vaccines according to the invention may either be prophylactic (i.e. to prevent infection) or therapeutic (i.e. to treat infection), but will typically be prophylactic.

Compositions may thus be pharmaceutically acceptable. They will usually include components in addition to the antigen(s) e.g. they typically include one or more pharmaceutical carrier(s) and/or excipient(s). A thorough discussion of such components is available in reference 252.

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To improve thermal stability, a composition may include a temperature protective agent. Further details of such agents are provided below.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below.

Thus the invention provides an immunogenic composition comprising a combination of: (1) a BAA antigen; and (2) an adjuvant, such as an aluminium hydroxide adjuvant (for example, one or more antigens may be adsorbed to aluminium hydroxide).

Adjuvants which may be used in compositions of the invention include, but are not limited to:

A. Mineral-Containing Compositions

Mineral containing compositions suitable for use as adjuvants in the invention include mineral salts, such as aluminium salts and calcium salts (or mixtures thereof). Calcium salts include calcium phosphate (e.g. the “CAP” particles disclosed in ref. 70). Aluminum salts include hydroxides, phosphates, sulfates, etc., with the salts taking any suitable form (e.g. gel, crystalline, amorphous, etc.). Adsorption to these salts is preferred (e.g. all antigens may be adsorbed). The mineral containing compositions may also be formulated as a particle of metal salt [71].

The adjuvants known as aluminum hydroxide and aluminum phosphate may be used. These names are conventional, but are used for convenience only, as neither is a precise description of the actual chemical compound which is present (e.g. see chapter 9 of reference 76). The invention can use any of the “hydroxide” or “phosphate” adjuvants that are in general use as adjuvants. The adjuvants known as “aluminium hydroxide” are typically aluminium oxyhydroxide salts, which are usually at least partially crystalline. The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt.

A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

Aluminium phosphate adjuvants generally have a PO₄/Al molar ratio between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

As shown below, adsorption of S. aureus protein antigens (except IsdA, Sta019 and Sta073) to an aluminium hydroxide adjuvant is advantageous, particularly in a multi-protein combination (in which all antigens may be adsorbed). A histidine buffer can usefully be included in such adjuvanted compositions.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

The invention can use a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The concentration of Al⁺⁺⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of 0.85 mg/dose is preferred.

B. Oil Emulsions

Oil emulsion compositions suitable for use as adjuvants in the invention include squalene-water emulsions, such as MF59 [Chapter 10 of ref. 76; see also ref. 72] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Various oil-in-water emulsion adjuvants are known, and they typically include at least one oil and at least one surfactant, with the oil(s) and surfactant(s) being biodegradable (metabolisable) and biocompatible. The oil droplets in the emulsion are generally less than 5 μm in diameter, and ideally have a sub-micron diameter, with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetraco sahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Preferred emulsion adjuvants have an average droplets size of <1 μm e.g. ≦750 nm, ≦500 nm, ≦400 mn, ≦300 nm, ≦250 nm, ≦220 nm, ≦200 nm, or smaller. These droplet sizes can conveniently be achieved by techniques such as microfluidisation.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [73-75], as described in more detail in Chapter 10 of ref. 76 and chapter 12 of ref. 77. The MF59 emulsion advantageously includes citrate ions e.g. 10 mM sodium citrate buffer.
An emulsion of squalene, a tocopherol, and polysorbate 80 (Tween 80). The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is preferably ≦1 as this provides a more stable emulsion. Squalene and Tween 80 may be present volume ratio of about 5:2 or at a weight ratio of about 11:5. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm. The emulsion may also include a 3-de-O-acylated monophosphoryl lipid A (3d-MPL). Another useful emulsion of this type may comprise, per human dose, 0.5-10 mg squalene, 0.5-11 mg tocopherol, and 0.1-4 mg polysorbate 80 [78].
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [79] (0.05-1% Thr-MDP, 5% squalane, 2.5% Pluronic L121 and 0.2% polysorbate 80). It can also be used without the Thr-MDP, as in the “AF” adjuvant [80] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [81]. The emulsion may also include one or more of: alditol; a cryoprotective agent (e.g. a sugar, such as dodecylmaltoside and/or sucrose); and/or an alkylpolyglycoside. The emulsion may include a TLR4 agonist [82]. Such emulsions may be lyophilized.
An emulsion of squalene, poloxamer 105 and Abil-Care [83]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 84, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 85, produced by addition of aliphatic amine to desacylsaponin via the carboxyl group of glucuronic acid), dimethyidioctadecylammonium bromide and/or N,N-dioctadecyl-N,N-bis(2-hydroxyethyl)propanediamine.
An emulsion in which a saponin (e.g. QuilA or QS21) and a sterol (e.g. a cholesterol) are associated as helical micelles [86].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [87].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [87].

In some embodiments an emulsion may be mixed with antigen extemporaneously, at the time of delivery, and thus the adjuvant and antigen may be kept separately in a packaged or distributed vaccine, ready for final formulation at the time of use. In other embodiments an emulsion is mixed with antigen during manufacture, and thus the composition is packaged in a liquid adjuvanted form. The antigen will generally be in an aqueous form, such that the vaccine is finally prepared by mixing two liquids. The volume ratio of the two liquids for mixing can vary (e.g. between 5:1 and 1:5) but is generally about 1:1. Where concentrations of components are given in the above descriptions of specific emulsions, these concentrations are typically for an undiluted composition, and the concentration after mixing with an antigen solution will thus decrease.

Where a composition includes a tocopherol, any of the α, β, γ, δ, ε or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in vaccines for use in elderly patients (e.g. aged 60 years or older) because vitamin E has been reported to have a positive effect on the immune response in this patient group [88]. They also have antioxidant properties that may help to stabilize the emulsions [89]. A preferred α-tocopherol is DL-α-tocopherol, and the preferred salt of this tocopherol is the succinate. The succinate salt has been found to cooperate with TNF-related ligands in vivo.

C. Saponin Formulations [Chapter 22 of Ref 76]

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref 90. Saponin formulations may also comprise a sterol, such as cholesterol [91].

Combinations of saponins and cholesterols can be used to form unique particles called immunostimulating complexs (ISCOMs) [chapter 23 of ref 76]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 91-93. Optionally, the ISCOMS may be devoid of additional detergent [94].

A review of the development of saponin based adjuvants can be found in refs. 95 & 96.

D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 97-102. Virosomes are discussed further in, for example, ref. 103

E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref 104. Such “small particles” of 3dMPL are small enough to be sterile filtered through a 0.22 μm membrane [104]. Other non-toxic LPS derivatives include monophosphoryl lipid A mimics, such as aminoalkyl glucosaminide phosphate derivatives e.g. RC-529 [105,106].

Lipid A derivatives include derivatives of lipid A from Escherichia coli such as OM-174. OM-174 is described for example in refs. 107 & 108.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 109, 110 and 111 disclose possible analog substitutions e.g. replacement of guanosine with 2′-deoxy-7-deazaguanosine. The adjuvant effect of CpG oligonucleotides is further discussed in refs. 112-117.

The CpG sequence may be directed to TLR9, such as the motif GTCGTT or TTCGTT [118]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 119-121. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 118 & 122-124.

A useful CpG adjuvant is CpG7909, also known as ProMune™ (Coley Pharmaceutical Group, Inc.). Another is CpG1826. As an alternative, or in addition, to using CpG sequences, TpG sequences can be used [125], and these oligonucleotides may be free from unmethylated CpG motifs. The immunostimulatory oligonucleotide may be pyrimidine-rich. For example, it may comprise more than one consecutive thymidine nucleotide (e.g. TTTT, as disclosed in ref 125), and/or it may have a nucleotide composition with >25% thymidine (e.g. >35%, >40%, >50%, >60%, >80%, etc.). For example, it may comprise more than one consecutive cytosine nucleotide (e.g. CCCC, as disclosed in ref 125), and/or it may have a nucleotide composition with >25% cytosine (e.g. >35%, >40%, >50%, >60%, >80%, etc.). These oligonucleotides may be free from unmethylated CpG motifs. Immunostimulatory oligonucleotides will typically comprise at least 20 nucleotides. They may comprise fewer than 100 nucleotides.

A particularly useful adjuvant based around immunostimulatory oligonucleotides is known as IC-31™ [126]. Thus an adjuvant used with the invention may comprise a mixture of (i) an oligonucleotide (e.g. between 15-40 nucleotides) including at least one (and preferably multiple) CpI motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′ (SEQ ID NO: 6). The polycationic polymer may be a peptide comprising 11-mer amino acid sequence KLKLLLLLKLK (SEQ ID NO: 7). The oligonucleotide and polymer can form complexes e.g. as disclosed in references 127 & 128.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 129 and as parenteral adjuvants in ref. 130. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 131-138. A useful CT mutant is or CT-E29H [139]. Numerical reference for amino acid substitutions is preferably based on the alignments of the A and B subunits of ADP-ribosylating toxins set forth in ref. 140, specifically incorporated herein by reference in its entirety.

F. Human Immunomodulators

Human immunomodulators suitable for use as adjuvants in the invention include cytokines, such as interleukins (e.g. IL-1, IL-2, IL-4, IL-5, IL-6, IL-7, IL-12 [141], etc.) [142], interferons (e.g. interferon-γ), macrophage colony stimulating factor, and tumor necrosis factor. A preferred immunomodulator is IL-12.

G. Bioadhesives and Mucoadhesives

Bioadhesives and mucoadhesives may also be used as adjuvants in the invention. Suitable Bioadhesives include esterified hyaluronic acid microspheres [143] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [144].

H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

I. Liposomes (Chapters 13 & 14 of Ref 76)

Examples of liposome formulations suitable for use as adjuvants are described in refs. 145-147.

J. Polyoxyethylene Ether and Polyoxyethylene Ester Formulations

Adjuvants suitable for use in the invention include polyoxyethylene ethers and polyoxyethylene esters [148]. Such formulations further include polyoxyethylene sorbitan ester surfactants in combination with an octoxynol [149] as well as polyoxyethylene alkyl ethers or ester surfactants in combination with at least one additional non-ionic surfactant such as an octoxynol [150]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

K. Phosphazenes

A phosphazene, such as poly[di(carboxylatophenoxy)phosphazene] (“PCPP”) as described, for example, in references 151 and 152, may be used.

L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

M. Imidazoquinolone Compounds.

Examples of imidazoquinolone compounds suitable for use adjuvants in the invention include Imiquimod (“R-837”) [153,154], Resiquimod (“R-848”) [155], and their analogs; and salts thereof (e.g. the hydrochloride salts). Further details about immunostimulatory imidazoquinolines can be found in references 156 to 160.

N. Substituted Ureas

Substituted ureas useful as adjuvants include compounds of formula I, II or III, or salts thereof:

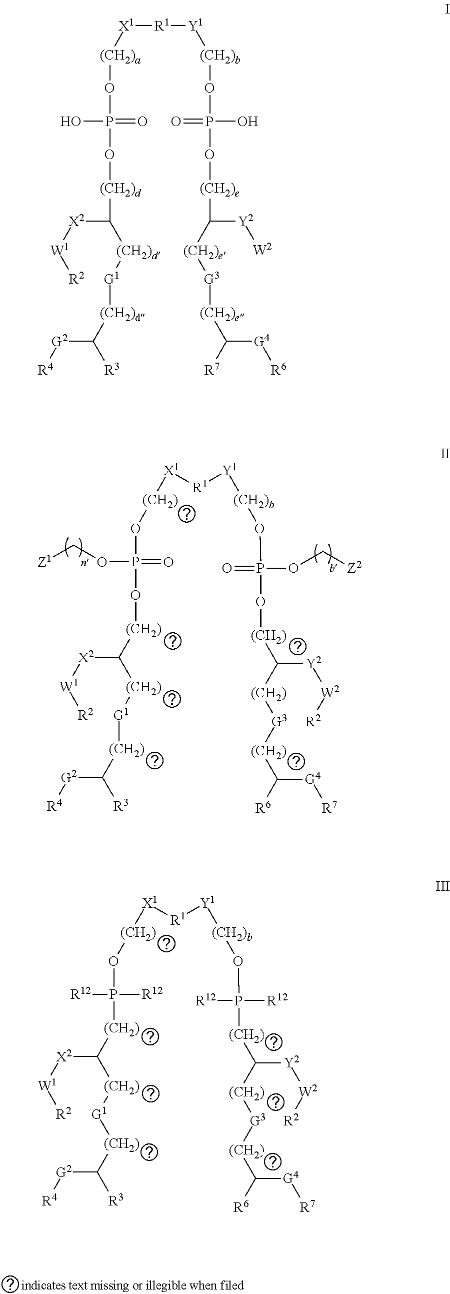

as defined in reference 161, such as ‘ER 803058’, ‘ER 803732’, ‘ER 804053’, ER 804058’, ‘ER 804059’, ‘ER 804442’, ‘ER 804680’, ‘ER 804764’, ER 803022 or ‘ER 804057’ e.g.:

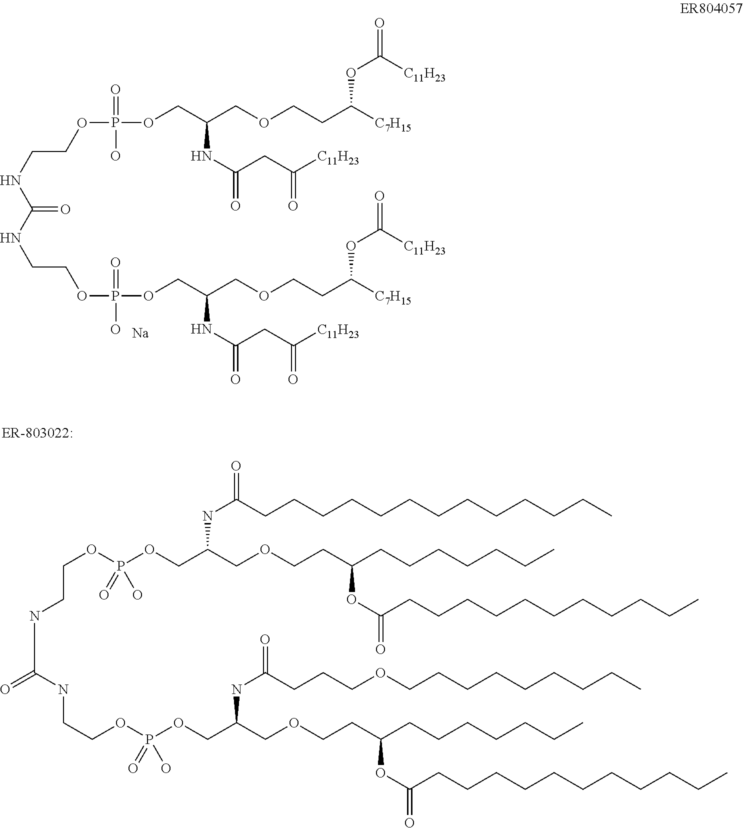

O. Further Adjuvants

Further adjuvants that may be used with the invention include:
Cyclic diguanylate (‘c-di-GMP’), which has been reported as a useful adjuvant for S. aureus vaccines [162].
A thiosemicarbazone compound, such as those disclosed in reference 163. Methods of formulating, manufacturing, and screening for active compounds are also described in reference 163. The thiosemicarbazones are particularly effective in the stimulation of human peripheral blood mononuclear cells for the production of cytokines, such as TNF-α.
A tryptanthrin compound, such as those disclosed in reference 164. Methods of formulating, manufacturing, and screening for active compounds are also described in reference 164. The thiosemicarbazones are particularly effective in the stimulation of human peripheral blood mononuclear cells for the production of cytokines, such as TNF-α.
A nucleoside analog, such as: (a) Isatorabine (ANA-245; 7-thia-8-oxoguanosine):

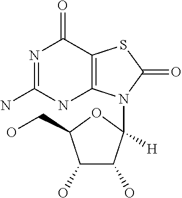 [SMILES: Nc1nc(=O)c2sc(=O)n([C@@H]3O[C@H](CO)[C@@H](O)[C@H]3O)c2[nH]1]

and prodrugs thereof; (b) ANA975; (c) ANA-025-1; (d) ANA380; (e) the compounds disclosed in references 165 to 167 Loxoribine (7-allyl-8-oxoguanosine) [168].
Compounds disclosed in reference 169, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds [170,171], Hydrapthalamide compounds, Benzophenone compounds, Isoxazole compounds, Sterol compounds, Quinazilinone compounds, Pyrrole compounds [172], Anthraquinone compounds, Quinoxaline compounds, Triazine compounds, Pyrazalopyrimidine compounds, and Benzazole compounds [173].
Compounds containing lipids linked to a phosphate-containing acyclic backbone, such as the TLR4 antagonist E5564 [174,175]:
A polyoxidonium polymer [176,177] or other N-oxidized polyethylene-piperazine derivative.
Methyl inosine 5′-monophosphate (“MIMP”) [178].
A polyhydroxlated pyrrolizidine compound [179], such as one having formula:

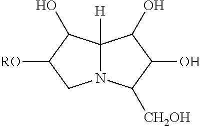

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.
A CD1d ligand, such as an a-glycosylceramide [180-187] (e.g. α-galactosylceramide), phytosphingosine-containing α-glycosylceramides, OCH, KRN7000 [(2S,3S,4R)-1-O-(α-D-galactopyranosyl)-2-(N-hexacosanoylamino)-1,3,4-octadecanetriol], CRONY-101, 3″-O-sulfo-galactosylceramide, etc.
A gamma inulin [188] or derivative thereof, such as algammulin.

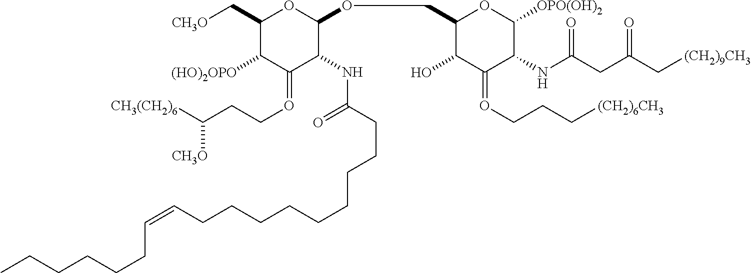

Adjuvant Combinations

The invention may also comprise combinations of one or more of the adjuvants identified above. For example, the following adjuvant compositions may be used in the invention: (1) a saponin and an oil-in-water emulsion [189]; (2) a saponin (e.g. QS21)+a non-toxic LPS derivative (e.g. 3 dMPL) [190]; (3) a saponin (e.g. QS21)+a non-toxic LPS derivative (e.g. 3 dMPL)+a cholesterol; (4) a saponin (e.g. QS21)+3 dMPL+IL-12 (optionally+a sterol) [191]; (5) combinations of 3 dMPL with, for example, QS21 and/or oil-in-water emulsions [192]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3 dMPL).

Other substances that act as immunostimulating agents are disclosed in chapter 7 of ref. 76.

The use of an aluminium hydroxide and/or aluminium phosphate adjuvant is particularly preferred, and antigens are generally adsorbed to these salts. Calcium phosphate is another preferred adjuvant. Other preferred adjuvant combinations include combinations of Th1 and Th2 adjuvants such as CpG & alum or resiquimod & alum. A combination of aluminium phosphate and 3 dMPL may be used.

The compositions of the invention may elicit both a cell mediated immune response as well as a humoral immune response. This immune response will preferably induce long lasting (e.g. neutralising) antibodies and a cell mediated immunity that can quickly respond upon exposure to pnuemococcus.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MEC Class I molecules.

CD4 T cells can express a CD4 co-receptor and are commonly referred to as T helper cells. CD4 T cells are able to recognize antigenic peptides bound to MEC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune resonse will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

Preferably, the invention includes a composition comprising a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

S. aureus infections can affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Where more than one antigen is included in a composition then two antigens may be present at the same dose as each other or at different doses.

As mentioned above, a composition may include a temperature protective agent, and this component may be particularly useful in adjuvanted compositions (particularly those containing a mineral adjuvant, such as an aluminium salt). As described in reference 193, a liquid temperature protective agent may be added to an aqueous vaccine composition to lower its freezing point e.g. to reduce the freezing point to below 0° C. Thus the composition can be stored below 0° C., but above its freezing point, to inhibit thermal breakdown. The temperature protective agent also permits freezing of the composition while protecting mineral salt adjuvants against agglomeration or sedimentation after freezing and thawing, and may also protect the composition at elevated temperatures e.g. above 40° C. A starting aqueous vaccine and the liquid temperature protective agent may be mixed such that the liquid temperature protective agent forms from 1-80% by volume of the final mixture. Suitable temperature protective agents should be safe for human administration, readily miscible/soluble in water, and should not damage other components (e.g. antigen and adjuvant) in the composition. Examples include glycerin, propylene glycol, and/or polyethylene glycol (PEG). Suitable PEGs may have an average molecular weight ranging from 200-20,000 Da. In a preferred embodiment, the polyethylene glycol can have an average molecular weight of about 300 Da (‘PEG-300’).

The invention provides an immunogenic composition comprising: (i) one or more antigen(s) selected from the first, second, third or fourth antigen groups; and (ii) a temperature protective agent. This composition may be formed by mixing (i) an aqueous composition comprising one or more antigen(s) selected from the first, second, third or fourth antigen groups, with (ii) a temperature protective agent. The mixture may then be stored e.g. below 0° C., from 0-20° C., from 20-35° C., from 35-55° C., or higher. It may be stored in liquid or frozen form. The mixture may be lyophilised. The composition may alternatively be formed by mixing (i) a dried composition comprising one or more antigen(s) selected from the first, second, third or fourth antigen groups, with (ii) a liquid composition comprising the temperature protective agent. Thus component (ii) can be used to reconstitute component (i).

Methods of Treatment, and Administration of Vaccines

The invention also provides a method for raising an immune response in a mammal comprising the step of administering an effective amount of an antigen, protein or immunogenic composition of the invention. The immune response is preferably protective and preferably involves antibodies and/or cell-mediated immunity. The method may raise a booster response.

The invention also provides a method for immunising a mammal against S. aureus, comprising the step of administering an effective amount of an antigen, protein or immunogenic composition of the invention.

The invention also provides the use of a BAA antigen in the manufacture of a medicament for immunising against S. aureus disease and/or infection.

The invention also provides a fusion protein or an immunogenic composition of the invention for use in therapy.

The invention also provides the use of a fusion protein of the invention in the manufacture of a medicament for immunising against S. aureus disease and/or infection.

The invention provides an anti-BAA antibody of the invention for use in therapy. The invention also provides the use of an anti-BAA antibody in the manufacture of a medicament for protecting and/or treating S. aureus disease and/or infection.

By raising an immune response in the mammal by these uses and methods, the mammal can be protected against S. aureus infection, including a nosocomial infection. More particularly, the mammal may be protected against a catheter related blood stream infection, skin infection, pneumonia, meningitis, osteomyelitis endocarditis, toxic shock syndrome, and/or septicaemia.

The invention also provides a delivery device pre-filled with an immunogenic composition of the invention.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc. Other mammals which can usefully be immunised according to the invention are cows, dogs, horses, and pigs.

One way of checking efficacy of therapeutic treatment involves monitoring S. aureus infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the antigens in the compositions of the invention after administration of the composition. Typically, antigen-specific serum antibody responses are determined post-immunisation but pre-challenge whereas antigen-specific mucosal antibody responses are determined post-immunisation and post-challenge.

Another way of assessing the immunogenicity of the compositions of the present invention is to express the proteins recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The efficacy of vaccine compositions can also be determined in vivo by challenging animal models of S. aureus infection, e.g., guinea pigs or mice, with the vaccine compositions. In particular, there are three useful animal models for the study of S. aureus infectious disease, namely: (i) the murine abscess model [194], (ii) the murine lethal infection model [194] and (iii) the murine pneumonia model [195]. The abscess model looks at abscesses in mouse kidneys after intravenous challenge. The lethal infection model looks at the number of mice which survive after being infected by a normally-lethal dose of S. aureus by the intravenous or intraperitoneal route. The pneumonia model also looks at the survival rate, but uses intranasal infection. A useful vaccine may be effective in one or more of these models. For instance, for some clinical situations it may be desirable to protect against pneumonia, without needing to prevent hematic spread or to promote opsonisation; in other situations the main desire may be to prevent hematic spread. Different antigens, and different antigen combinations, may contribute to different aspects of an effective vaccine.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, 1-5 years old, 5-15 years old, 15-55 years old, or at least 55 years old. Preferred patients for receiving the vaccines are the elderly (e.g. ≧50 years old, ≧60 years old, and preferably ≧65 years), the young (e.g. ≦5 years old), hospitalised patients, healthcare workers, armed service and military personnel, pregnant women, the chronically ill, or immunodeficient patients. The vaccines are not suitable solely for these groups, however, and may be used more generally in a population.

Vaccines produced by the invention may be administered to patients at substantially the same time as (e.g. during the same medical consultation or visit to a healthcare professional or vaccination centre) other vaccines e.g. at substantially the same time as an influenza vaccine, a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc. Further non-staphylococcal vaccines suitable for co-administration may include one or more antigens listed on pages 33-46 of reference 58.

Nucleic Acid Immunisation

The immunogenic compositions described above include polypeptide antigens from S. aureus. In all cases, however, the polypeptide antigens can be replaced by nucleic acids (typically DNA) encoding those polypeptides, to give compositions, methods and uses based on nucleic acid immunisation. Nucleic acid immunisation is now a developed field (e.g. see references 196 to 203 etc.).

The nucleic acid encoding the immunogen is expressed in vivo after delivery to a patient and the expressed immunogen then stimulates the immune system. The active ingredient will typically take the form of a nucleic acid vector comprising: (i) a promoter; (ii) a sequence encoding the immunogen, operably linked to the promoter; and optionally (iii) a selectable marker. Preferred vectors may further comprise (iv) an origin of replication; and (v) a transcription terminator downstream of and operably linked to (ii). In general, (i) & (v) will be eukaryotic and (iii) & (iv) will be prokaryotic.

Preferred promoters are viral promoters e.g. from cytomegalovirus (CMV). The vector may also include transcriptional regulatory sequences (e.g. enhancers) in addition to the promoter and which interact functionally with the promoter. Preferred vectors include the immediate-early CMV enhancer/promoter, and more preferred vectors also include CMV intron A. The promoter is operably linked to a downstream sequence encoding an immunogen, such that expression of the immunogen-encoding sequence is under the promoter's control.

Where a marker is used, it preferably functions in a microbial host (e.g. in a prokaryote, in a bacteria, in a yeast). The marker is preferably a prokaryotic selectable marker (e.g. transcribed under the control of a prokaryotic promoter). For convenience, typical markers are antibiotic resistance genes.

The vector of the invention is preferably an autonomously replicating episomal or extrachromosomal vector, such as a plasmid.

The vector of the invention preferably comprises an origin of replication. It is preferred that the origin of replication is active in prokaryotes but not in eukaryotes.

Preferred vectors thus include a prokaryotic marker for selection of the vector, a prokaryotic origin of replication, but a eukaryotic promoter for driving transcription of the immunogen-encoding sequence. The vectors will therefore (a) be amplified and selected in prokaryotic hosts without polypeptide expression, but (b) be expressed in eukaryotic hosts without being amplified. This arrangement is ideal for nucleic acid immunization vectors.

The vector of the invention may comprise a eukaryotic transcriptional terminator sequence downstream of the coding sequence. This can enhance transcription levels. Where the coding sequence does not have its own, the vector of the invention preferably comprises a polyadenylation sequence. A preferred polyadenylation sequence is from bovine growth hormone.

The vector of the invention may comprise a multiple cloning site

In addition to sequences encoding the immunogen and a marker, the vector may comprise a second eukaryotic coding sequence. The vector may also comprise an IRES upstream of said second sequence in order to permit translation of a second eukaryotic polypeptide from the same transcript as the immunogen. Alternatively, the immunogen-coding sequence may be downstream of an IRES.

The vector of the invention may comprise unmethylated CpG motifs e.g. unmethylated DNA sequences which have in common a cytosine preceding a guanosine, flanked by two 5′ purines and two 3′ pyrimidines. In their unmethylated form these DNA motifs have been demonstrated to be potent stimulators of several types of immune cell.

Vectors may be delivered in a targeted way. Receptor-mediated DNA delivery techniques are described in, for example, references 204 to 209. Therapeutic compositions containing a nucleic acid are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g. for enhancing or inhibiting levels of the encoded gene product) and efficacy of transformation and expression are considerations which will affect the dosage required for ultimate efficacy. Where greater expression is desired over a larger area of tissue, larger amounts of vector or the same amounts re-administered in a successive protocol of administrations, or several administrations to different adjacent or close tissue portions may be required to effect a positive therapeutic outcome. In all cases, routine experimentation in clinical trials will determine specific ranges for optimal therapeutic effect.

Vectors can be delivered using gene delivery vehicles. The gene delivery vehicle can be of viral or non-viral origin (see generally references 210 to 213).

Viral-based vectors for delivery of a desired nucleic acid and expression in a desired cell are well known in the art. Exemplary viral-based vehicles include, but are not limited to, recombinant retroviruses (e.g. references 214 to 224), alphavirus-based vectors (e.g. Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532); hybrids or chimeras of these viruses may also be used), poxvirus vectors (e.g. vaccinia, fowlpox, canarypox, modified vaccinia Ankara, etc.), adenovirus vectors, and adeno-associated virus (AAV) vectors (e.g. see refs. 225 to 230). Administration of DNA linked to killed adenovirus [231] can also be employed.

Non-viral delivery vehicles and methods can also be employed, including, but not limited to, polycationic condensed DNA linked or unlinked to killed adenovirus alone [e.g. 231], ligand-linked DNA [232], eukaryotic cell delivery vehicles cells [e.g. refs. 233 to 237] and nucleic charge neutralization or fusion with cell membranes. Naked DNA can also be employed. Exemplary naked DNA introduction methods are described in refs. 238 and 239. Liposomes (e g. immunoliposomes) that can act as gene delivery vehicles are described in refs. 240 to 244. Additional approaches are described in references 245 & 246.

Further non-viral delivery suitable for use includes mechanical delivery systems such as the approach described in ref. 246. Moreover, the coding sequence and the product of expression of such can be delivered through deposition of photopolymerized hydrogel materials or use of ionizing radiation [e.g. refs. 247 & 248l ]. Other conventional methods for gene delivery that can be used for delivery of the coding sequence include, for example, use of hand-held gene transfer particle gun [249] or use of ionizing radiation for activating transferred genes [247 & 248].

Delivery DNA using PLG {poly(lactide-co-glycolide)} microparticles is a particularly preferred method e.g. by adsorption to the microparticles, which are optionally treated to have a negatively-charged surface (e.g. treated with SDS) or a positively-charged surface (e.g. treated with a cationic detergent, such as CTAB).

Detection and Diagnostic Methods

The invention provides a method for detecting a S. aureus bacterium in a sample. The method can involve detecting the presence or absence of a BAA antigen or of nucleic acid encoding a BAA antigen. The method can be used for microbiological testing, clinical or non-clinical diagnosis, etc. Detection of the antigen may involve e.g. contacting the sample with an anti-BAA antibody, such as a labelled anti-BAA antibody. Detection of the nucleic acid antigen may involve any convenient method e.g. based on nucleic acid hybridisation, such as by using northern or southern blots, nucleic acid microarrays or ‘gene chips’, amplification reactions (e.g. PCR, SDA, SSSR, LCR, TMA, NASBA, etc.).

The invention also provides a method for detecting if a patient has been infected with S. aureus, comprising a step of detecting in a sample taken from the patient the presence or absence of an anti-BAA antibody. Detection of the antigen may involve, for example, contacting the sample with a BAA antigen e.g. an immobilised BAA antigen.

Presence of the BAA antigen, or of nucleic acid encoding the BAA antigen, or of an anti-BAA antigen, indicates the presence of S. aureus in the sample, and in particular indicates the presence of a “TW” MRSA strain and/or a type ST-239 MRSA strain. In a clinical diagnostic setting, therefore, the results of the method may be used to educate or dictate a therapeutic strategy for a patient e.g. a choice of antibiotics, etc.

The invention also provides a process for detecting a BAA antigen, comprising the steps of: (a) contacting an anti-BAA antibody with a biological sample under conditions suitable for the formation of an antibody-antigen complexes; and (b) detecting the complexes.

The invention also provides a process for detecting anti-BAA antibodies, comprising the steps of: (a) contacting a BAA antigen with a biological sample (e.g. a blood or serum sample) under conditions suitable for the formation of an antibody-antigen complexes; and (b) detecting the complexes.

The invention provides a process for detecting a BAA-encoding nucleic acid, comprising the steps of (a) contacting a nucleic probe according to the invention with a biological sample under hybridising conditions to form duplexes; and (b) detecting said duplexes.

The invention also provides a kit comprising primers (e.g. PCR primers) for amplifying a template sequence contained within a S. aureus bacterium BAA nucleic acid sequence, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label (e.g. a fluorescent label).

The invention also provides a kit comprising first and second single-stranded oligonucleotides which allow amplification of a S. aureus BAA template nucleic acid sequence contained in a single- or double-stranded nucleic acid (or mixture thereof), wherein: (a) the first oligonucleotide comprises a primer sequence which is substantially complementary to said template nucleic acid sequence; (b) the second oligonucleotide comprises a primer sequence which is substantially complementary to the complement of said template nucleic acid sequence; (c) the first oligonucleotide and/or the second oligonucleotide comprise(s) sequence which is not complementary to said template nucleic acid; and (d) said primer sequences define the termini of the template sequence to be amplified. The non-complementary sequence(s) of feature (c) are preferably upstream of (i.e. 5′ to) the primer sequences. One or both of these (c) sequences may comprise a restriction site [e.g. ref.250] or a promoter sequence [e.g. 251]. The first oligonucleotide and/or the second oligonucleotide may include a detectable label (e.g. a fluorescent label).

General

The practice of the present invention will employ, unless otherwise indicated, conventional methods of chemistry, biochemistry, molecular biology, immunology and pharmacology, within the skill of the art. Such techniques are explained fully in the literature. See, e.g., references 252-259, etc.

Where the invention concerns an “epitope”, this epitope may be a B-cell epitope and/or a T-cell epitope. Such epitopes can be identified empirically (e.g. using PEPSCAN [260,261] or similar methods), or they can be predicted (e.g. using the Jameson-Wolf antigenic index [262], matrix-based approaches [263], MAPITOPE [264], TEPITOPE [265,266], neural networks [267], OptiMer & EpiMer [268, 269], ADEPT [270], Tsites [271], hydrophilicity [272], antigenic index [273] or the methods disclosed in references 274-278, etc.). Epitopes are the parts of an antigen that are recognised by and bind to the antigen binding sites of antibodies or T-cell receptors, and they may also be referred to as “antigenic determinants”.

Where an antigen “domain” is omitted, this may involve omission of a signal peptide, of a cytoplasmic domain, of a transmembrane domain, of an extracellular domain, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 279. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 280.

### BRIEF DESCRIPTION OF DRAWINGS

FIG. 1 shows a comparison of adherence to fibronectin at 37° C. of four L. lactis bacteria, measured as OD_{570 nm}. The bars show, from left-to-right, a positive control bacterium, a TOPO strain expressing BAA, an infusion strain expressing BAA, and a control strain transformed with empty vector.

### MODES FOR CARRYING OUT THE INVENTION

In 2007 an unusual strain of MRSA was reported as a cause of vascular-access device (VAD)-related bacteraemia in a London intensive care unit (ICU) [3]. Genetic analysis of the strain using a microarray provided evidence that this “TW” strain represents a version of ST239 epidemic clones previously reported in the UK. The strain has acquired all detectable mobile genetic elements associated with virulence which are variably expressed by other epidemic ST239 clones. A cohort analysis of all patients acquiring MRSA on the ICU showed that the adjusted hazard ratio for bacteremia in patients acquiring TW MRSA was 4.5 times higher than those acquiring non-TW MRSA strains. TW MRSA was also significantly more likely to be isolated from vascular catheters than non-TW MRSA strains.

Clinical observations suggested that TW's ability to cause bacteremia is due to an enhanced adhesive capacity to extracellular matrix proteins which are adsorbed in vivo onto the surface of vascular catheters.

Adhesion of MRSA strains to fibrinogen, fibronectin, elastin and laminin was assessed in 96-well plates. Plates were coated with 10 μg/ml protein solutions at 4° C. overnight. Wells were rinsed in PBS then incubated in 100 μl 2% BSA at 37° C. for one hour to block non-specific binding. Overnight bacterial cell suspension was adjusted with RPMI-1640 medium to an OD_{600 nm} of 0.1, inoculated in microtitre plate, and incubated for 2 hours at 37° C. The wells were washed in PBS thrice to remove non-adherent bacteria. Adherent bacteria were fixed in 100 μl 25% formaldehyde then stained with 100 μl 0.1% crystal violet for 15 minutes and rinsed under running tap water After airdrying the stained adherent bacterial film adsorbance was measured with a microplate reader. All assay plates included an appropriate positive control where possible and sterile TSB lacking bacteria as a negative control. The OD_{570 nm} for an isolate was the reading after subtraction of the background (same plate negative control OD_{570 nm}). Each assay was performed on 30 occasions.

TW MRSA demonstrated greater adhesion to fibronectin, fibrinogen and elastin than a related ST-239 strain (EMRSA-1), ST-22 and ST-36, indicating that adhesion to one or more of these extracellular matrix proteins is a likely mechanism for adherence to catheters.

Genomic sequencing of TW MRSA identified a large phage with homology to a phage previously identified in S. epidermidis (RP62a) [6]. The TW phage contains nucleotide sequence SEQ ID NO: 8 encoding a putative 21 kD surface-expressed LP×TG adhesin “BAA” with the following amino acid sequence (SEQ ID NO: 1):

MKKSKVLATTTLAGALLFTGVGATRNAHAADYVNDSNVRDYAKNAIGSKYTEAGSVSIGGHGGGVTDKDNGVK
DNGDYYTVIFSGEKDNGVGAAKVYKDGRIEAQTPRDANNVLTIDAPNNTQSTDNTQAATNNNTTATTDNNVST
QENNTQSTQSTQTNEAQTTTKALPETGGQSNSGLVTIIASVLLAAGSLLAFRRTSNSK

BAA has not previously been reported in any S. aureus strain and neither has it been associated with an ability to cause bacteremia. BAA is present (detected by PCR) in all TW MRSA strains which were tested (80 in total), 27% of other ST-239 strains, 10% of other bacteremic sporadic gentamicin resistant MRSA and MSSA strains and 41% of bacteremic gentamicin resistant S. epidermidis strains. Its presence in S. epidermidis suggests that it may also be involved in bacteremia due to coagulase negative staphylococcal strains.

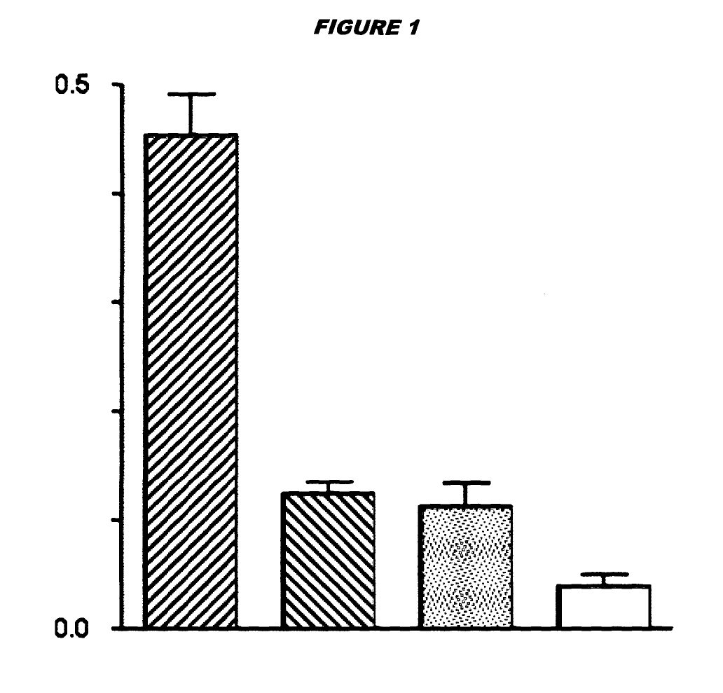

Lactococcus lactis subsp. cremoris was transfected with pkS80 encoding BAA. Positive transformants were screened for by touch PCR. Presence of the cloned construct was confirmed by sequencing of positive transformant plasmid preparation. Stable transformants of both pkS80/BAA and empty vector control were assessed for binding to fibronectin as described above. There was specific binding to fibronectin with vectors constructed using both TOPO and infusion methodologies (FIG. 1).

BAA from S. epidermidis is disclosed as SesD in reference 7. SesD is expressed and exposed to the human immune system, and human serum showed high titers against SesD. The SesD sequence in reference 7 is 95% identical to SEQ ID NO: 1 herein and so the immunological results seen with SesD can also be expected for BAA antigens of the invention.

Thus TW MRSA carries a novel phage-encoded predicted surfaced-expressed adhesin (BAA) and in vitro demonstrates enhanced binding to both fibrinogen, fibronectin, and elastin compared with other endemic and related strains. Preliminary experiments demonstrate that transfection of BAA into Lactococcus strains confers a fibronectin binding phenotype. The proposal is that BAA is responsible for the enhanced catheter-related bacteremic phenotype of TW MRSA observed in the clinical setting and therefore given the broad distribution of ST-239 strains worldwide, that BAA is a plausible vaccine target to prevent bacteremia.

It will be understood that the invention has been described by way of example only and modifications may be made whilst remaining within the scope and spirit of the invention.

### REFERENCES

[1] Sheridan (2009) Nature Biotechnology 27:499-501.

[2] Kuklin et al. (2006) Infect Immun. 74(4):2215-23.

[3] Edgeworth et al. (2007) Clin Infect Dis 44:493-501.

[4] Bowden et al. (2005) Microbiol 151:1453-64.

[5] Söderquist et al. (2009) J Med Microbiol 58:1395-7.

[6] Gill et al. (2005) J Bacteriol 187:2426-38.

[7] WO2007/060546.

[8] Needleman & Wunsch (1970) J. Mol. Biol. 48, 443-453.

[9] Rice et al. (2000) Trends Genet 16:276-277.

[10] WO2007/113222.

[11] WO2005/009379.

[12] WO2009/029132.

[13] WO2008/079315.

[14] WO2005/086663.

[15] WO2005/115113.

[16] WO2006/033918.

[17] WO2006/078680.

[18] WO2007/113224.

[19] WO98/10788.

[20] WO2007/053176.

[21] O'Brien et al. (2000) J Dairy Sci 83:1758-66.

[22] Research Disclosure, 453077 (January 2002).

[23] EP-A-0372501.

[24] EP-A-0378881.

[25] EP-A-0427347.

[26] WO93/17712.

[27] WO94/03208.

[28] WO98/58668.

[29] EP-A-0471177.

[30] WO91/01146.

[31] Falugi et al. (2001) Eur J Immunol 31:3816-3824.

[32] Baraldo et al. (2004) Infect Immun 72(8):4884-7.

[33] EP-A-0594610.

[34] Ruan et al. (1990) J Immunol 145:3379-3384.

[35] WO00/56360.

[36] Kuo et al. (1995) Infect Immun 63:2706-13.

[37] Michon et al. (1998) Vaccine. 16:1732-41.

[38] WO02/091998.

[39] WO01/72337.

[40] WO00/61761.

[41] WO00/33882

[42] U.S. Pat. No. 4,761,283.

[43] U.S. Pat. No. 4,356,170.

[44] U.S. Pat. No. 4,882,317.

[45] U.S. Pat. No. 4,695,624.

[46] Mol. Immunol., 1985, 22, 907-919

[47] EP-A-0208375.

[48] Bethell G. S. et al., J. Biol. Chem., 1979, 254, 2572-4

[49] Hearn M. T. W., J. Chromatogr., 1981, 218, 509-18

[50] WO00/10599.

[51] Geyer et al., Med. Microbiol. Immunol, 165: 171-288 (1979).

[52] U.S. Pat. No. 4,057,685.

[53] U.S. Pat. Nos. 4,673,574; 4,761,283; 4,808,700.

[54] U.S. Pat. NO. 4,459,286.

[55] U.S. Pat. No. 4,965,338.

[56] U.S. Pat. No. 4,663,160.

[57] WO2007/000343.

[58] WO2008/019162.

[59] Winter et al., (1991) Nature 349:293-99

[60] U.S. Pat. No. 4,816,567.

[61] Inbar et al., (1972) Proc. Natl. Acad. Sci. U.S.A. 69:2659-62.

[62] Ehrlich et al., (1980) Biochem 19:4091-96.

[63] Huston et al., (1988) Proc. Natl. Acad. Sci. U.S.A. 85:5897-83.

[64] Pack et al., (1992) Biochem 31, 1579-84.

[65] Cumber et al., (1992) J. Immunology 149B, 120-26.

[66] Riechmann et al., (1988) Nature 332, 323-27.

[67] Verhoeyan et al., (1988) Science 239, 1534-36.

[68] GB 2,276,169.

[69] U.S. Pat. No. 5,707,829

[70] U.S. Pat. No. 6,355,271.

[71] WO00/23105.

[72] WO90/14837.

[73] WO90/14837.

[74] Podda & Del Giudice (2003) Expert Rev Vaccines 2:197-203.

[75] Podda (2001) Vaccine 19: 2673-2680.

[76] Vaccine Design: The Subunit and Adjuvant Approach (eds. Powell & Newman) Plenum Press 1995 (ISBN 0-306-44867-X).

[77] Vaccine Adjuvants: Preparation Methods and Research Protocols (Volume 42 of Methods in Molecular Medicine series). ISBN: 1-59259-083-7. Ed. O'Hagan.

[78] WO2008/043774.

[79] Allison & Byars (1992) Res Immunol 143:519-25.

[80] Hariharan et al. (1995) Cancer Res 55:3486-9.

[81] US-2007/014805.

[82] US-2007/0191314.

[83] Suli et al. (2004) Vaccine 22(25-26):3464-9.

[84] WO95/11700.

[85] U.S. Pat. No. 6,080,725.

[86] WO2005/097181.

[87] WO2006/113373.

[88] Han et al. (2005) Impact of Vitamin E on Immune Function and Infectious Diseases in the Aged at Nutrition, Immune functions and Health EuroConference, Paris, 9-10 Jun. 2005.

[89] U.S. Pat. No. 6,630,161.

[90] U.S. Pat. No. 5,057,540.

[91] WO96/33739.

[92] EP-A-0109942.

[93] WO96/11711.

[94] WO00/07621.

[95] Barr et al. (1998) Advanced Drug Delivery Reviews 32:247-271.

[96] Sjolanderet et al. (1998) Advanced Drug Delivery Reviews 32:321-338.

[97] Niikura et al. (2002) Virology 293:273-280.

[98] Lenz et al. (2001) J Immunol 166:5346-5355.

[99] Pinto et al. (2003) J Infect Dis 188:327-338.

[100] Gerber et al. (2001) J Virol 75:4752-4760.

[101] WO03/024480.

[102] WO03/024481.

[103] Gluck et al. (2002) Vaccine 20:B10-B16.

[104] EP-A-0689454.

[105] Johnson et al. (1999) Bioorg Med Chem Lett 9:2273-2278.

[106] Evans et al. (2003) Expert Rev Vaccines 2:219-229.

[107] Meraldi et al. (2003) Vaccine 21:2485-2491.

[108] Pajak et al. (2003) Vaccine 21:836-842.

[109] Kandimalla et al. (2003) Nucleic Acids Research 31:2393-2400.

[110] WO02/26757.

[111] WO99/62923.

[112] Krieg (2003) Nature Medicine 9:831-835.

[113] McCluskie et al. (2002) FEMS Immunology and Medical Microbiology 32:179-185.

[114] WO98/40100.

[115] U.S. Pat. No. 6,207,646.

[116] U.S. Pat. No. 6,239,116.

[117] U.S. Pat. No. 6,429,199.

[118] Kandimalla et al. (2003) Biochemical Society Transactions 31 (part 3):654-658.

[119] Blackwell et al. (2003) J Immunol 170:4061-4068.

[120] Krieg (2002) Trends Immunol 23:64-65.

[121] WO01/95935.

[122] Kandimalla et al. (2003) BBRC 306:948-953.

[123] Bhagat et al. (2003) BBRC 300:853-861.

[124] WO03/035836.

[125] WO01/22972.

[126] Schellack et al. (2006) Vaccine 24:5461-72.

[127] Kamath et al. (2008) Eur J Immunol 38:1247-56.

[128] Riedl et al. (2008) Vaccine 26:3461-8.

[129] WO95/17211.

[130]WO98/42375.

[131] Beignon et al. (2002) Infect Immun 70:3012-3019.

[132] Pizza et al. (2001) Vaccine 19:2534-2541.

[133] Pizza et al. (2000) Int J Med Microbiol 290:455-461.

[134] Scharton-Kersten et al. (2000) Infect Immun 68:5306-5313.

[135] Ryan et al. (1999) Infect Immun 67:6270-6280.

[136] Partidos et al. (1999) Immunol Lett 67:209-216.

[137] Peppoloni et al. (2003) Expert Rev Vaccines 2:285-293.

[138] Pine et al. (2002) J Control Release 85:263-270.

[139] Tebbey et al. (2000) Vaccine 18:2723-34.

[140] Domenighini et al. (1995) Mol Microbiol 15:1165-1167.

[141] WO99/40936.

[142] WO99/44636.

[143] Singh et al] (2001) J Cont Release 70:267-276.

[144] WO99/27960.

[145] U.S. Pat. No. 6,090,406.

[146] U.S. Pat. No. 5,916,588.

[147] EP-A-0626169.

[148] WO99/52549.

[149] WO01/21207.

[150] WO01/21152.

[151] Andrianov et al. (1998) Biomaterials 19:109-115.

[152] Payne et al. (1998) Adv Drug Delivery Review 31:185-196.

[153] U.S. Pat. No. 4,680,338.

[154] U.S. Pat. No. 4,988,815.

[155] WO92/15582.

[156] Stanley (2002) Clin Exp Dermatol 27:571-577.

[157] Wu et al. (2004) Antiviral Res. 64(2):79-83.

[158] Vasilakos et al. (2000) Cell Immunol. 204(1):64-74.

[159] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.

[160] Jones (2003) Curr Opin Investig Drugs 4:214-218.

[161] WO03/011223.

[162] Hu et al. (2009) Vaccine 27:4867-73.

[163] WO2004/060308.

[164] WO2004/064759.

[165] U.S. Pat. No. 6,924,271.

[166] US2005/0070556.

[167] U.S. Pat. No. 5,658,731.

[168] U.S. Pat. No. 5,011,828.

[169] WO2004/87153.

[170] U.S. Pat. No. 6,605,617.

[171] WO02/18383.

[172] WO2004/018455.

[173] WO03/082272.

[174] Wong et al. (2003) J Clin Pharmacol 43(7):735-42.

[175] US2005/0215517.

[176] Dyakonova et al. (2004) Int Immunopharmacol 4(13):1615-23.

[177] FR-2859633.

[178] Signorelli & Hadden (2003) Int Immunopharmacol 3(8):1177-86.

[179] WO2004/064715.

[180] De Libero et al, Nature Reviews Immunology, 2005, 5: 485-496

[181] U.S. Pat. No. 5,936,076.

[182] Oki et al, J. Clin. Investig., 113: 1631-1640

[183] US2005/0192248

[184] Yang et al, Angew. Chem. Int. Ed., 2004, 43: 3818-3822

[185] WO2005/102049

[186] Goff et al, J. Am. Chem., Soc., 2004, 126: 13602-13603

[187] WO03/105769

[188] Cooper (1995) Pharm Biotechnol 6:559-80.

[189] WO99/11241.

[190] WO94/00153.

[191] WO98/57659.

[192] European patent applications 0835318, 0735898 and 0761231.

[193] WO2006/110603.

[194] Stranger-Jones et al. (2006) PNAS USA 103:16942-7.

[195] Wardenburg et al. (2007) Infect Immun 75:1040-4.

[196] Donnelly et al. (1997) Annu Rev Immunol 15:617-648.

[197] Strugnell et al. (1997) Immunol Cell Biol 75(4):364-369.

[198] Cui (2005) Adv Genet 54:257-89.

[199] Robinson & Tones (1997) Seminars in Immunol 9:271-283.

[200] Brunham et al. (2000) J Infect Dis 181 Suppl 3:S538-43.

[201] Svanholm et al. (2000) Scand J Irnmunol 51(4):345-53.

[202] DNA Vaccination—Genetic Vaccination (1998) eds. Koprowski et al. (ISBN 3540633928).

[203] Gene Vaccination: Theory and Practice (1998) ed. Raz (ISBN 3540644288).

[204] Findeis et al., Trends Biotechnol. (1993) 11:202

[205] Chiou et al. (1994) Gene Therapeutics: Methods And Applications Of Direct Gene Transfer. ed. Wolff

[206] Wu et al., J. Biol. Chem. (1988) 263:621

[207] Wu et al., J. Biol. Chem. (1994) 269:542

[208] Zenke et al., Proc. Natl. Acad. Sci. (USA) (1990) 87:3655

[209] Wu et al., J. Biol. Chem. (1991) 266:338

[210] Jolly, Cancer Gene Therapy (1994) 1:51

[211] Kimura, Human Gene Therapy (1994) 5:845

[212] Connelly, Human Gene Therapy (1995) 1:185

[213] Kaplitt, Nature Genetics (1994) 6:148

[214] WO 90/07936.

[215] WO 94/03622.

[216] WO 93/25698.

[217] WO 93/25234.

[218] U.S. Pat. No. 5,219,740.

[219] WO 93/11230.

[220] WO 93/10218.

[221] U.S. Pat. No. 4,777,127.

[222] GB Patent No. 2,200,651.

[223] EP-A-0345242.

[224] WO 91/02805.

[225] WO 94/12649.

[226] WO 93/03769.

[227] WO 93/19191.

[228] WO 94/28938.

[229] WO 95/11984.

[230] WO 95/00655.

[231] Curiel, Hum. Gene Ther. (1992) 3:147

[232] Wu, J. Biol. Chem. (1989) 264:16985

[233] U.S. Pat. No. 5,814,482.

[234] WO 95/07994.

[235] WO 96/17072.

[236] WO 95/30763.

[237] WO 97/42338.

[238] WO 90/11092.

[239] U.S. Pat. No. 5,580,859

[240] U.S. Pat. No. 5,422,120

[241] WO 95/13796.

[242] WO 94/23697.

[243] WO 91/14445.

[244] EP-0524968.

[245] Philip, Mol. Cell Biol. (1994) 14:2411

[246] Woffendin, Proc. Natl. Acad. Sci. (1994) 91:11581

[247] U.S. Pat. No. 5,206,152.

[248] WO 92/11033.

[249] U.S. Pat. No. 5,149,655.

[250] EP-B-0509612.

[251] EP-B-0505012.

[252] Gennaro (2000) Remington: The Science and Practice of Pharmacy. 20th edition, ISBN: 0683306472.

[253] Methods In Enzymology (S. Colowick and N. Kaplan, eds., Academic Press, Inc.)

[254] Handbook of Experimental Immunology, Vols. I-IV (D. M. Weir and C. C. Blackwell, eds, 1986, Blackwell Scientific Publications)

[255] Sambrook et al. (2001) Molecular Cloning: A Laboratory Manual, 3rd edition (Cold Spring Harbor Laboratory Press).

[256] Handbook of Surface and Colloidal Chemistry (Birdi, K. S. ed., CRC Press, 1997)

[257] Ausubel et al. (eds) (2002) Short protocols in molecular biology, 5th edition (Current Protocols).

[258] Molecular Biology Techniques: An Intensive Laboratory Course, (Ream et al., eds., 1998, Academic Press)

[259] PCR (Introduction to Biotechniques Series), 2nd ed. (Newton & Graham eds., 1997, Springer Verlag)

[260] Geysen et al. (1984) PNAS USA 81:3998-4002.

[261] Carter (1994) Methods Mol Biol 36:207-23.

[262] Jameson, B A et al. 1988, CABIOS 4(1):181-186.

[263] Raddrizzani & Hammer (2000) Brief Bioinform 1(2):179-89.

[264] Bublil et al. (2007) Proteins 68(1):294-304.

[265] De Lalla et al. (1999) J. Immunol. 163:1725-29.

[266] Kwok et al. (2001) Trends Immunol 22:583-88.

[267] Brusic et al. (1998) Bioinformatics 14(2):121-30

[268] Meister et al. (1995) Vaccine 13(6):581-91.

[269] Roberts et al. (1996) AIDS Res Hum Retroviruses 12(7):593-610.

[270] Maksyutov & Zagrebelnaya (1993) Comput Appl Biosci 9(3):291-7.

[271] Feller & de la Cruz (1991) Nature 349(6311):720-1.

[272] Hopp (1993) Peptide Research 6:183-190.

[273] Welling et al. (1985) FEBS Lett. 188:215-218.

[274] Davenport et al. (1995) Immunogenetics 42:392-297.

[275] Tsurui & Takahashi (2007) J Pharmacol Sci. 105(4):299-316.

[276] Tong et al. (2007) Brief Bioinform. 8(2):96-108.

[277] Schirle et al. (2001) J Immunol Methods. 257(1-2):1-16.

[278] Chen et al. (2007) Amino Acids 33(3):423-8.

[279] Current Protocols in Molecular Biology (F. M. Ausubel et al., eds., 1987) Supplement 30

[280] Smith & Waterman (1981) Adv. Appl. Math. 2: 482-489.

1. A BAA antigen for use in immunising against S. aureus disease and/or infection.

2. A fusion protein comprising a BAA antigen polypeptide sequence and at least one further S. aureus antigen polypeptide sequence.

3. An immunogenic composition comprising a BAA antigen and at least one further S. aureus antigen.

4. An immunogenic composition comprising a BAA antigen and at least one non-S. aureus antigen.

5. An immunogenic composition comprising a combination of: (1) a BAA antigen; and (2) an adjuvant.

6. A method for detecting the presence or absence of a S. aureus bacterium in a sample, comprising detecting a BAA antigen, or nucleic acid encoding a BAA antigen, in the sample.

7. The antigen of claim 1, wherein the BAA antigen can elicit an antibody which recognises the S. aureus protein having amino acid sequence SEQ ID NO: 1.

8. The antigen of claim 1, wherein the BAA antigen comprises an amino acid sequence: (a) having 80% or more identity to SEQ ID NO: 1; and/or (b) comprising an epitope of SEQ ID NO: 1.

9. The antigen of claim 1, wherein the BAA antigen comprises an amino acid sequence: (a) having 50% or more identity to SEQ ID NO: 1; and/or (b) comprising a fragment of at least 7 consecutive amino acids of SEQ ID NO: 1.

10. An anti-BAA antibody.

11. The antibody of claim 10, which is a monoclonal antibody which recognises the S. aureus protein having amino acid sequence SEQ ID NO: 1.

12. The antibody of claim 10, for use in protecting against or treating S. aureus infection and/or disease.


SEQUENCE LISTING (8 sequences)

SEQ ID NO: 1 - protein, 204 aa, Staphylococcus aureus
MKKSKVLATTTLAGALLFTGVGATHNAHAADYVNDSNVRDYAKNAIGSKYTEAGSVSIGGHGGGVTDKDNGVKDNGDYYTVIFSGEKDNGVGAAKVYKDGRIEAQTPRDANNVLTIDAPNNTQSTDNTQAATNNNTTATTDNNVSTQENNTQSTQSTQTNEAQTTTKALPETGGQSNSGLVTIIASVLLAAGSLLAFRRTSNSK

SEQ ID NO: 2 - protein, 6 aa, Artificial Sequence
  Linker Peptide
GSGGGG

SEQ ID NO: 3 - protein, 8 aa, Artificial Sequence
  Linker Peptide
GSGSGGGG

SEQ ID NO: 4 - protein, 6 aa, Artificial Sequence
  Linker Peptide
ASGGGS

SEQ ID NO: 5 - protein, 6 aa, Artificial Sequence
  Histidine tag
HHHHHH

SEQ ID NO: 6 - DNA, 26 bp, Artificial Sequence
  Immunostimulatory oligonucleotide
ncncncncncncncncncncncncnc

SEQ ID NO: 7 - protein, 11 aa, Artificial Sequence
  Polycationic oligopeptide
KLKLLLLLKLK

SEQ ID NO: 8 - DNA, 615 bp, Staphylococcus aureus
atgaaaaaatctaaagttttagcaacaactacattagcgggagcattattatttacaggtgtaggagctacacataatgcacatgcagctgattatgtaaatgactcaaatgttagagattatgctaaaaatgctattggttcaaaatatactgaagcgggttctgtatcaattggaggacacggtggtggcgttactgataaagacaatggagtaaaagacaatggtgactattatactgttattttttctggtgaaaaagacaatggtgtaggtgctgcaaaagtttataaagatggacgtattgaagctcagactcctagagacgcaaacaatgtattaacaattgacgcaccaaacaatacacaaagcactgacaatactcaagcagcaacaaataacaacactactgctacaactgataacaatgtttctactcaagaaaacaatacacaatcaactcaatcaactcaaactaacgaagctcaaacaacaacaaaagcgttaccagaaacaggcggacaatctaattctggtttagtaacaatcattgcttcagtattattagcagctggttcattattagcatttagacgtacttctaattctaaataa